(19) United States
(12) Patent Application Publication
(10) Pub. No.: US 2019/0016660 A1
(43) Pub. Date: Jan. 17, 2019

(54) PROCESS FOR PREPARING ALKYL SALICYLIC ACID AND PRODUCTS THEREOF

(71) Applicant: SI GROUP, INC., Schenectady, NY (US)

(72) Inventors: Milan Knezevic, Schenectady, NY (US); Cornelius Haase, Schenectady, NY (US); Joseph Zampella, Niskayuna, NY (US); Scott Smith, Schenectady, NY (US); Todd Makenzie, Schenectady, NY (US)

(21) Appl. No.: 16/031,844

(22) Filed: Jul. 10, 2018

Related U.S. Application Data
(60) Provisional application No. 62/531,023, filed on Jul. 11, 2017.

(51) Int. Cl. C07C 51/353; C07C 65/10

(52) U.S. Cl. CPC C07C 51/353; C07C 65/10

(57) ABSTRACT

This invention relates to a process for preparing an alkylsalicylic acid. The process comprises reacting salicylic acid with an olefin having at least four carbon atoms at a temperature ranging from about 50° C. to about 200° C. in the presence of an arylsulfonic acid-containing catalyst (such as para-toluene sulfonic acid), to produce an alkylsalicylic acid. The resulting alkylsalicylic acid has various applications such as a food preservative, an oil field chemical for oil recovery, and a component in a color toner agent for electrophotography.

### CROSS REFERENCE TO RELATED APPLICATIONS

This application claims the benefit of priority to U.S. Provisional Patent Application Ser. No. 62/531,023, filed Jul. 11, 2017, which is hereby incorporated by reference in its entirety.

### FIELD OF THE INVENTION

This invention generally relates to a process for preparing alkylsalicylic acid and the products thereof.

### BACKGROUND

Alkylsalicylic acids have widespread applications in various fields. They can be used as an oil field chemical for oil recovery, or as an additive in lubricating oils. Smaller alkylsalicylic acids, such as 5-t-butylsalicylic acid and 3,5-di-tert-butylsalicylic acid, can be used as anti-oxidants in the food industry or as a component in a color toner agent for electrophotography.

Alkylsalicylic acids have been made through the alkylation of salicylic acid with an olefin using sulfuric acid, (poly)phosphoric acid, alkylsulfonic acid, acidic ion exchange resin, or acidic clay as a catalyst. However, there are problems associated with using these catalysts. For instance, when sulfuric acid is used to catalyze the alkylation of salicylic acid with alkenes, certain competing reactions can take place, such as formation of esters when adding an alkene to sulfuric acid, and sulfonation of the aromatic ring. When using alkylsulfonic acid, such as methane sulfonic acid (see U.S. Pat. No. 7,045,654), the reaction time is relatively long (usually about 24 hours or longer) and the reaction condition is relatively harsh (typically at 120° C.). Chinese Patent No. 103508881B discusses the use of benzene sulfonic acid as the catalyst to prepare alkyl salicylic acid. When using benzene sulfonic acid as the catalyst, the reaction condition is relatively harsh (typically at 120° C. or above; and argon needs to be introduced into the reaction system), and the olefin conversion rate is low (76.2%-89.6%), i.e., at least 10 mol % residual olefin would be present in the alkylsalicylic product. Chinese Patent Application Publication No. 104098466A also similarly uses benzene sulfonic acid as the catalyst to prepare alkyl salicylate. There, the reaction setup is sophisticated and requires at least a specific vessel that functions as a water trap for the alkylation reaction to constantly remove water from the reaction system. Also, the olefin conversion rate is not discussed.

Therefore, there remains a need in the art to develop a process to prepare alkylsalicylic acids more easily and efficiently, with a better conversion rate, and under milder reaction conditions, to produce a better quality product (e.g., relatively higher acid number and relatively less residual olefin in the product and having recyclability of the catalyst. This invention answers that need and others.

### SUMMARY OF THE INVENTION

One aspect of the invention relates to a process for preparing an alkylsalicylic acid comprising reacting salicylic acid with an olefin having at least four carbon atoms at a temperature ranging from about 50° C. to about 200° C. in the presence of an arylsulfonic acid-containing catalyst, to produce an alkylsalicylic acid with a residual olefin of less than about 2 wt %. The molar ratio of the olefin to salicylic acid is about 1:1 to about 1.3:1.

Another aspect of the invention relates to a process for preparing an alkylsalicylic acid comprising reacting salicylic acid with an olefin having at least four carbon atoms at a temperature ranging from about 50° C. to about 200° C. in the presence of para-toluene sulfonic acid, to produce an alkylsalicylic acid with a total acid number no less than 90.

The disclosed invention demonstrates an alkylation process for salicylic acid, which can be conducted at low reaction temperatures in short reaction times, producing cleaner products with higher acid numbers. Anhydrous para-toluenesulfonic acid, when used as the catalyst, can be precipitated as a crystalline monohydrate, which allows it to be removed by filtration, leaving low or even residual amounts of catalyst in the final product. Once filtered off, the catalyst can be recycled for the use in subsequent salicylic acid alkylation reactions.

### DETAILED DESCRIPTION OF THE INVENTION

This invention relates to a process for preparing an alkylsalicylic acid. The process comprises reacting salicylic acid with an olefin having at least four carbon atoms at a temperature ranging from about 50° C. to about 200° C. in the presence of an arylsulfonic acid-containing catalyst (such as para-toluene sulfonic acid), to produce an alkylsalicylic acid. The alkylsalicylic acid can have a residual olefin of less than about 2 wt %, and/or a total acid number no less than 90. There are many benefits associated with using para-toluene sulfonic acid (“PTSA”), particularly in-situ prepared, anhydrous para-toluene sulfonic acid. For instance, when the process uses para-toluene sulfonic acid, it allows the completion of the reaction in a relatively shorter time, e.g., no more than 10 hours, at a relatively mild temperature (at about 100° C.) compared to the same process using methane sulfonic acid as the catalyst (typically about 24 hours of reaction time at about 120° C.), to produce an alkylsalicylic acid with low residual olefin content and a high acid number. There is no aqueous washing necessary when using para-toluene sulfonic acid as the catalyst. Additionally, recyclability of the catalyst is relatively easy when using para-toluene sulfonic acid as the catalyst. This is because the main liquid catalyst layer can be easily separated from the organic reaction mass layer, and the residual catalyst in the organic reaction mass can be precipitated as the PTSA monohydrate, which can be easily removed and recovered by filtration. This filtration saves a water wash step that may be present with the previously described processes, and allows an easy recovery of otherwise lost catalyst. The combined PTSA monohydrate and the catalyst layer can be recycled after a drying step.

Commercially available salicylic acid can be used with or without further purification.

The olefins may be linear or branched olefins having, e.g., 4 to 60 carbon atoms, 4 to 50 carbon atoms, 4 to 36 carbon atoms, 4 to 24 carbon atoms, or 4 to 12 carbon atoms. Typically, the olefin used to prepare the alkylsalicylic acid is an α-olefin, such as a linear α-olefin. An exemplary olefin is C₁₆ α-olefin, such as C₁₆ linear α-olefin. Other suitable olefins include, but are not limited to, isobutylene, propylene trimer, propylene tetramer, 1-hexene, 1-octene, 1-decene, 1-dodecene, 1-tetradecene, 1-octadecene, 1-eicosene, 1-docosene, and 1-tetracosene. The olefin used may be a mixture of two or more different olefins.

The amounts of olefins used in preparing the alkylsalicylic acid may vary in a wide range. For instance, the molar ratio of the olefin to salicylic acid can range from about 0.7: 1 to about 1.5:1, from about 1.1 to about 1.3:1, or about 1.05:1 to about 1.2:1. The olefin can be added in single portion, in two or more portions over time, or gradually during the reaction. It is advantageous to add the olefin to the reaction in two or more portions over time or gradually during the reaction, e.g., adding several portions over a period of time (e.g., about 30 minutes to about 20 hours, or about 2 to about 6 hours), continuously (e.g. dropwise) or in batches, while maintaining the reaction temperature, because this slow, portioned addition at different time points changes the kinetics of the reaction and promotes a better consumption of the salicylic acid by the olefin, thus contributing to the high yield and high acid number of the final product. This way, the quality of the final product is significantly improved without lengthening the total reaction time and without using harsher reaction conditions.

The catalyst used is an arylsulfonic acid. Exemplary catalysts include para-toluene sulfonic acid, xylene sulfonic acid, naphthalene sulfonic acid, phenol sulfonic acid, or combinations thereof. In one embodiment, the catalyst is para-toluene sulfonic acid. It is desirable that the catalyst contains little water, or is anhydrous, e.g., having less than about 5% water, having less than about 2% water, or having no more than 0.5% water.

The amounts of catalyst used in preparing the alkylsalicylic acid may vary widely. For instance, the molar ratio of the arylsulfonic acid to salicylic acid can range from about 0.5:1 to about 1.5:1, or from about 0.7:1 to about 1:1.

Commercially available arylsulfonic acid-containing catalyst can be used. When commercially available arylsulfonic acid is used, a drying step may be needed to remove the excess water that may be present in the arylsulfonic acid. An organic solvent, such as n-heptane or n-octane can be used to dry the arylsulfonic acid-containing catalyst (e.g., para-toluene sulfonic acid), and can subsequently be removed under vacuum condition. Typically, the drying agent is chosen so that the boiling point of the drying agent is above the melting point of the arylsulfonic acid monohydrate (e.g., PTSA monohydrate's melting point is 103-106° C.), thus, keeping the reaction mass liquid during the drying stage. Care is needed when using n-heptane as a drying agent with PTSA, as this mixture can cause partial solidification in the reaction mass when reaching the para-toluene sulfonic acid monohydrate stage, although this solid partition can disappear as more water is removed. When using n-octane as a drying agent, the temperature in the drying pot at the final drying stage should be closely monitored so that it does not go over the boiling point of the drying agent, which can cause possible side reactions and darkening of the catalyst.

Often times, using in-situ prepared anhydrous arylsulfonic acid in the reaction process is desirable. In-situ para-toluene sulfonic acid can be prepared by a process comprising reacting toluene with a sulfonating agent at a temperature ranging from about 0° C. to about 200° C.; and removing water from the reaction mixture. Exemplary sulfonating agents include concentrated sulfuric acid, fuming sulfuric acid, and sulfur trioxide. The reaction temperature of preparing para-toluene sulfonic acid can change depending on the sulfonating agents used. For instance, when using concentrated sulfuric acid as the sulfonating agent, the reaction temperature can range from about 50° C. to about 200° C.; when the sulfonating agent is sulfur trioxide, the reaction temperature can range from about 0° C. to about 30° C.; and when the sulfonating agent is fuming sulfuric acid, the reaction temperature can range from about 0° C. to about 70° C. The in-situ para-toluene sulfonic acid can then be used directly in the reaction of alkylation of salicylic acid without further purification. The preparations of other anhydrous arylsulfonic acids are similar to the preparation of para-toluene sulfonic acid.

The temperature of the alkylation reaction can range from about 50° C. to about 180° C., from about 60° C. to about 120° C., or from about 90° C. to about 110° C. For a C₁₆ α-olefin, the temperature typically ranges from about 90° C. to about 110° C., for instance, at about 100° C.

Typically, no solvent is used for the alkylation reaction.

The reaction of alkylation of salicylic acid can be complete within 10 hours, although a reaction time longer than 10 hours is permissible.

One advantage of this invention is the ease of recyclability of the catalyst. An aqueous wash of the reaction mixture is not needed. The arylsulfonic acid catalyst, such as para-toluene sulfonic acid, can be easily recycled because para-toluene sulfonic acid forms a monohydrate with water and can precipitate out of the organic reaction mixture layer. It can then be filtered out and used in the alkylation reaction.

Accordingly, some embodiments of the invention relate to a further process involving, after the alkylation reaction, separating the resulting alkylsalicylic acid by liquid-liquid extraction, in which an organic solvent is used to create a phase split between the organic layer containing mainly the alkylsalicylic acid product and a liquid layer containing mainly the residual arylsulfonic acid catalyst. The arylsulfonic acid catalyst can then be recovered from both the organic layer and the liquid layer to be recycled back into the reaction.

For instance, to recycle para-toluene sulfonic acid, after the reacting step, an organic solvent can be added to the reaction mixture to separate the catalyst layer from the reaction mixture layer. Suitable organic solvents include aromatic solvents, such as light naphtha, or alkane solvents, such as heptane or octane. After the separation of the catalyst layer from the reaction mixture layer, at least 70% of the para-toluene sulfonic acid catalyst can be recovered through this step. The catalyst contained in the separated catalyst layer can then be dried and recycled. The recycled, dried catalyst can be added directly back into the alkylation reaction.

The reaction mixture layer also contains the arylsulfonic acid catalyst. To further recover the arylsulfonic acid catalyst in the reaction mixture layer, water can be added in an amount sufficient to form an arylsulfonic acid monohydrate (e.g., para-toluene sulfonic acid monohydrate) precipitant by precipitating the remaining catalyst contained in the reaction mixture layer. The amount of water added to the reaction mixture layer is predetermined according to the estimated molar amount of arylsulfonic acid catalyst, wherein an equivalent molar amount of water is added to form arylsulfonic acid monohydrate. Examples 3 and 4 provide exemplary calculations for determining the amount of water added to the reaction mixture layer to form the para-toluene sulfonic acid monohydrate precipitant. The arylsulfonic acid monohydrate precipitant can then be recovered by filtration. After this precipitating step, the catalyst is typically sufficiently removed and the reaction mixture layer does not need to be further washed with water for removing additional catalyst. The precipitated catalyst can then be dried and recycled. The recycled, dried catalyst can be added directly back into the alkylation reaction.

Another aspect of the invention relates to the alkylsalicylic acid product prepared by the process described herein. The alkylsalicylic acid prepared by the process described here has many advantages, such as a high total acid number (TAN) and low residual olefin.

### Calculation of Total Acid Number (TAN):

The TAN is one measure for the quality of the alkylsalicylic acid product. The value of the TAN depends on the molecular weight of the olefin used (i.e., the number of the carbon atoms of the olefin), the molar ratio between the olefin and salicylic acid, the decarboxylation of salicylic acid, and possible ester formation of salicylic acid. A higher of the latter two will diminish the acidity of the system, while a higher of the former will “dilute” the acidity.

The theoretical acid number is determined as follows: the weight of the starting mass and the amount of salicylic acid in this starting mass are used to determine the amount of salicylic acid per gram of reaction mass. The molar amount of salicylic acid per gram of reaction mass is calculated and then the obtained number is multiplied by the molar mass of potassium hydroxide. The resulting number represents the grams of KOH necessary to neutralize one gram of the sample. The TAN is the milligrams of KOH necessary to neutralize one gram of the sample.

For example, if the reaction mass was 437.9 g reaction mass (277.0 g olefin and 160.9 g salicylic acid—1.165 mole salicylic acid), which means 0.002660 mole salicylic acid per gram of reaction mass. Then, if 0.1493 g KOH was needed to titrate 1 gram of the reaction mass, the theoretical TAN is 149.3 mg KOH/g.

As shown in the above determination of the theoretical total acid number, the value of the actual TAN can vary depending on the olefin used to make the alkylsalicylic acid and the molar ratio between the olefin and salicylic acid used to make the alkylsalicylic acid. The actual TAN typically is close to the theoretical TAN as determined above.

For the current disclosure, the TAN can be no less than 90, no less than 100, no less than 110, no less than 115, no less than 120, no less than 125, no less than 135, no less than 140, no less than 150, no less than 160, no less than 170, no less than 180, no less than 190, not less than 200, no less than 210, no less than 220, no less than 230, no less than 240, no less than 250, no less than 260, no less than 270, or no less than 280. The residual olefin is no more than about 5 wt %, or no more than about 2 wt %.

The examples help illustrate these properties. For instance, Examples 1 and 2 below illustrate when using a molar ratio of C₁₆-α-olefin to salicylic acid of 1.2:1, the final alkylsalicylic acid had an acid number of 129, 3.8 wt % unreacted salicylic acid, and 1.9 wt % unreacted C₁₆-olefin in the final product. Using the recycled PTSA catalyst at the same molar ratio of C₁₆-α-olefin to salicylic acid, under the same reaction condition, produced a similar high quality product with an acid number of 126.1, 3.6 wt % unreacted salicylic acid, and 1.5 wt % unreacted C₁₆-olefin in the final product. Examples 3 and 4 below illustrate when using a molar ratio of C₁₆-α-olefin to salicylic acid of 1.06:1, the final alkylsalicylic acid had an acid number of 140.7, 4.62 wt % unreacted salicylic acid, and 1.34 wt % unreacted C₁₆-olefin in the final product. Using the recycled PTSA catalyst at the same molar ratio of C₁₆-α-olefin to salicylic acid, under the same reaction condition, produced a similar high quality product with an acid number of 143.5, 5.15 wt % unreacted salicylic acid, and 1.44 wt % unreacted C₁₆-olefin in the final product.

The resulting alkylsalicylic acids have widespread applications in various fields. For instance, the alkylsalicylic acids can undergo an overbasing reaction and be used as intermediates in the preparation of lubricating oil additives. Any methods of overbasing alkylsalicylic acids well-known to one skilled in the art can be used herein. For example, the alkylsalicylic acids can be further reacted with a metal base (e.g., an alkali metal or an alkaline earth metal base, or a mixture of the two), in the presence of a solvent at elevated temperature. The base may take the form of the oxide or the hydroxide, e.g., slaked lime (i.e., calcium hydroxide). The amount of base added should be sufficient to provide an overbased salt, i.e., one in which the ratio of the number of equivalents of the metal moiety to the number of equivalents of the alkyl salicylic acid moiety is usually greater than about 1.2, and can be as high as 4.5 or greater. The metal base may be added either in a single addition or portioned additions during the reaction. The finished lubricating oil may also contain effective amounts of one or more other types of conventional lubricating oil additives, e.g., viscosity index improvers, anti-wear agents, antioxidants, dispersants, rust inhibitor, pour-point depressants, and combinations thereof.

Some embodiments of the invention also relate to using the resulting alkylsalicylic acids, such as 5-t-butylsalicylic acid and 3,5-di-tert-butylsalicylic acid, in an anti-oxidant composition in the food industry; or using the resulting alkylsalicylic acids, such as 5-t-butylsalicylic acid and 3,5-di-tert-butylsalicylic acid in a color toner composition for electrophotography.

### EXAMPLES

The following examples are given as particular embodiments of the invention and to demonstrate the practice and advantages thereof. It is to be understood that the examples are given by way of illustration and are not intended to limit the specification or the claims that follow in any manner.

### Example 1

### Alkylating Salicylic Acid with Self-Made PTSA as the Catalyst

PTSA was prepared by reacting toluene in the presence of sulfuric acid under conditions known to one skilled in the art to prepare an in-situ PTSA for subsequent use in the alkylation reaction.

A 500 ml three neck flask with a thermocouple, mechanical stirrer, and condenser was charged with 120 g of the in-situ prepared para-toluenesulfonic acid (“PTSA”; containing 0.4 wt % water). In a separate flask, a suspension of 247.6 g C₁₆-α-olefin and 127.0 g salicylic acid (1.2 molar equivalent of the olefin) was prepared.

The PTSA (120 g) was warmed, and then 372.2 g of the slurry was added to the PTSA. The stirring rate was increased to ˜370-390 rpm and the heating temperature was increased to 100° C. After maintaining at this temperature for about 10 hours, stirring was switched off and a sample of the upper part of the reaction mass was taken. It contained 3.86 wt % salicylic acid and 1.66 wt % unconverted olefin. From the initial total load of 492.2 g added to the flask, after 10 hours, 488.7 g was left in the flask.

The reaction mass was then poured into 151 g heptane. This led to a phase split into two layers: an upper organic layer containing mainly the reaction mass and a lower liquid layer containing mainly the residual acidic catalyst. A 101.8 g lower acidic catalyst layer was recovered.

At room temperature, 3.6 g water was added to the upper organic layer. The water associated with the remaining PTSA in the organic layer and formed PTSA monohydrate, which was easily filtered off. The white, crystalline filter cake (PTSA monohydrate) was washed with a total of 49.2 g heptane, and the washed organic solvent was combined with the organic reaction mass layer.

The combined organic layers were then concentrated under a reduced pressure to produce the final product in a yield of 96.5 wt %, with the final product having 3.8 wt % salicylic acid, 1.9 wt % C₁₆-olefin, 0.4 wt % heptane, and a total acid number of 129.0.

### Example 2

### Alkylating Salicylic Acid with Recycled PTSA as the Catalyst

The catalyst layers and the PTSA filter cake recovered from the organic layer from Example 1 were combined and dried. Subsequently, the recycled PTSA was used in the alkylation reaction. The alkylation reaction procedures were the same as those described in Example 1. The molar ratio of the recycled PTSA to salicylic acid and the molar ratio of salicylic acid to the C₁₆-α-olefin used in this example were the same as those in Example 1.

The reaction produced a final product in a yield of 97.9 wt %, with the final product having 3.6 wt % salicylic acid, 1.5 wt % C₁₆-olefin, 1.0 wt % heptane, and a total acid number of 126.1.

### Example 3

### Alkylation of Salicylic Acid with PTSA as the Catalyst

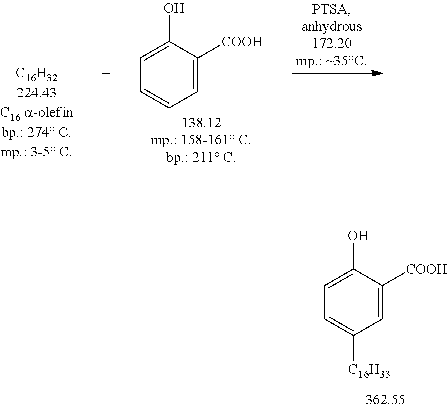

Salicylic acid can sublime in the upper parts of the reaction flasks. Therefore, the size of the reaction flask and the amount of reactants added were adjusted so that the flask was as full as possible, to minimize sublimation by rinsing down sublimate through agitation.

The reaction was conducted in a 500 ml four-neck flask, equipped with mechanical stirrer (PTFE blade, glass rod), thermocouple, an opening toward a condenser, and addition funnel. A Dean-Stark trap was incorporated in the reaction system to dry the PTSA prior to the alkylation reaction.

Commercially available aqueous PTSA (33.3% water) with high purity (<0.02% remaining sulfuric acid) (Dynachem Inc.) was used. PTSA was dried with n-octane (boiling point: 125-127° C.) as a drying agent because of n-octane's boiling point and speed of water removal. The boiling point of n-octane is above the melting point of the PTSA monohydrate (melting point: 103-106° C.), thus, keeping the reaction mass liquid during the drying stage.

### Drying Step:

180.5 g of the above aqueous PTSA and 160.6 g n-octane were added and heated. The mixture started to boil at 105.5° C. The drying step took about 4.5 hours and produced 59.2 g water. The final pot temperature was 129.1° C. Afterwards, octane was removed under vacuum (vacuum conditions: 40 Torr at 100° C. for 20-30 minutes). The final acid contained 9302 ppm sulfate and 2.05% n-octane.

### Reaction Step:

117.5 g of the PTSA catalyst prepared above was used for the reaction. 124.2 g salicylic acid (0.8992 mole) and 49.3 g C₁₆-α-olefin (0.2197 mole; 0.244 molar equivalent of salicylic acid) were added, and the slurry was heated to 101.2° C. At this point, 165.7 g C₁₆-α-olefin (0.7383 mole; 0.821 molar equivalent of salicylic acid) was added through the addition funnel over a course of 4.5 hours while maintaining the temperature. Total C₁₆-linear-α-olefin added was 215.0 g (0.9580 mole; 1.065 molar equivalent of salicylic acid). At the end of each portioned addition of olefin, the sublimed salicylic acid was transferred back into the reaction mass in one hour and two hours, respectively, after the start of the olefin addition by heating with a heat gun. Three hours later, no significant amount of additionally formed salicylic acid was observed. The reaction was stopped after a total of ten hours, and the flask content weighed 456.3 gram. Compared to the total added mass of 456.7 g, the mass loss was only 0.4 g.

Among the resulting reaction mass, 8.4 g was taken as a sample and 4.3 g was sticking to the flask. As a result, 443.6 g reaction mass was further processed. The reaction mass was poured into 135.8 g n-heptane. This led to a phase split of two layers, and the lower catalyst layer (108.5 g) settled out of the heptane layer.

A predetermined amount of water (2.64 g) was added at room temperature to the upper organic layer, and the resultant slurry was mixed. The slurry was allowed to sit for 5 minutes to form PTSA monohydrate, and then was filtered through a Buechner filter (regular filter paper). The obtained filter cake was washed with 100-200 ml additional heptane (so that the residual product in the cake may be extracted). The filter cake was dried for approximately 20-30 minutes, and 29.8 g filter cake was obtained. This material was substantially, but not completely dry.

The clear filtrate was concentrated under vacuum until a pressure of 40 Torr at ˜100° C. was reached and maintained for 20-30 minutes to produce 299.8 g final product. Together with the initial sample (8.4 g) and the content sticking to the flask (4.3 g), the rough isolated yield was 312.5 g (92.13 wt %).

The final product contained: sulfur, 1388 ppm; heptane/octane: 0.07 wt %; salicylic acid: 4.62 wt %; olefin: 1.34 wt %; PTSA: 1058 ppm; dialkyl region: 14.4 wt % (area% at 302 nm). Total acid number (TAN) for the final product was 140.7 mg KOH/g. The theoretical TAN was 148.7.

### Calculation of the Amount of Water Needed to Form PTSA Monohydrate

Based on the understanding that the lower catalyst layer contains 85% PTSA, 6% salicylic acid, and 9% alkylated salicylic acid product (based on the determination of the weight percentages of PTSA, the alkyl salicylic acid, and salicylic acid in this layer via HPLC), the 108.5 g lower catalyst layer would contain 92.2 g PTSA. Accordingly, the upper reaction mass layer would contain 25.3 g PTSA (0.1468 mole), which is the difference between the starting mass of PTSA (117.5 g) and the remaining PTSA in the lower catalyst layer (92.2 g) after the reaction. Therefore, 2.64 g of water could be added to the upper reaction mass layer to convert 0.1468 mole PTSA into PTSA monohydrate.

### Example 4

### Alkylating Salicylic Acid with Recycled PTSA as the Catalyst

### Drying Step:

106.0 g PTSA out of the 108.5 g lower catalyst layer from Example 3 was combined with the 29.8 g PTSA monohydrate filter cake from Example 3, added together with 137.0 g n-octane, and heated to reflux. The drying procedure took about 3 hours, and ˜2 g water was removed. A final temperature of 127.4° C. was reached. The octane was removed under reduced pressure (vacuum conditions: 40 Torr at 100° C. for 20-30 minutes). 128.6 g dried, recycled PTSA catalyst was obtained containing 0.17% water.

The PTSA content of the 128.6 g dried, recycled PTSA catalyst was determined by the following calculations. 106.0 g lower catalyst layer contained 85% PTSA (90.1 g), 6% salicylic acid (6.36 g), and 9% alkylated salicylic acid (9.54 g) (see discussions in Example 3). The filter cake from the upper layer then contained 22.6 g PTSA (i.e., the weight difference between the weight of the obtained dried recycled PTSA, 128.6 g, and the weight of the lower catalyst layer, 106.0 g). The combined recycled catalyst would then contain 112.7 g PTSA, 6.36 g salicylic acid, and 9.54 g alkylated salicylic acid. The amount of PTSA was a little less than the amount of PTSA (117.5 g) used in Example 3 and, thus, fresh/dry PTSA (9.5 g; see the reaction step below) was added in the reaction step.

### Reaction Step:

128.6 g recycled PTSA catalyst (containing 112.7 g PTSA, 6.36 g salicylic acid, and 9.54 g alkylated salicylic acid) was combined with 9.5 g fresh PTSA catalyst (dried with heptane) and used for this reaction. The total amount of PTSA catalyst used in this experiment was 122.2 g PTSA (0.7096 mole).

123.5 g fresh salicylic acid (0.8942 mole) and 51.0 g C₁₆-α-olefin (0.2272 mole; 0.257 molar equivalent of salicylic acid) were added to the catalyst layer. The total amount of salicylic acid in this experiment was 129.9 g (0.9402 mole), including the 6.36 g salicylic acid contained in the recycled PTSA catalyst layer (see the calculation above). The slurry was heated to 100.1° C. At this point, another portion of 172.3 g C₁₆-α-olefin (0.7677 mole; 0.817 molar equivalent of salicylic acid) was added through the addition funnel over a course of 4.5 hours while maintaining the temperature. Total C₁₆-linear-α-olefin added was 223.3 g (0.9950 mole; 1.058 molar equivalent of salicylic acid). The sublimed salicylic acid was transferred back to the reaction mass after each portioned addition of olefin. This periodic, slow addition of olefin changes the kinetics of the reaction and promotes better consumption of the salicylic acid by the olefin, thus contributing to the high yield and high TAN of the final product.

The reaction was stopped after a total of ten hours, the flask content weighed 484.6 g (mass loss of 6.7 g). The reaction mass was poured into 134.9 g n-heptane. Among the 484.6 g reaction mass, 4.8 g was taken as sample and 4.0 g was sticking to the flask. Therefore, 475.8 g reaction mass was subsequently processed. A 110.7 g lower catalyst layer settled out of the heptane layer.

A predetermined amount of water (2.94 g) was added at about 25° C. to the upper organic layer, and the resultant slurry was mixed. The slurry was allowed to sit for 5 minutes and was filtered through a Buchner funnel to form PTSA monohydrate. The obtained filter cake was washed with 100-200 ml additional heptane. The filter cake was vacuum-dried for 20-30 minutes. 29.5 g filter cake was obtained.

The clear filtrate was concentrated under vacuum until a pressure of about 40 Torr at about 100° C. was reached and maintained for 20-30 minutes to produce 337.9 g final product. Together with the initial sample (4.8 g) and the content sticking to the flask (4.0 g), the rough isolated yield was 342.7 g (98.2%).

The recovered lower catalyst layer contained alkylated salicylic acid product and salicylic acid (85% PTSA, 6% salicylic acid, and 9% alkylsalicylic acid).

The final product in this recycled catalyst reaction contained: sulfur, 1338 ppm; heptane/octane: 0.19 wt %; salicylic acid: 5.15 wt %; olefin: 1.44 wt %; PTSA: 1945 ppm; dialkyl region: 15.0 wt % (area% at 302 nm). TAN for the final product was 143.5 mg KOH/g. The theoretical TAN was 149.4.

### Calculation of the Amount of Water Needed to Form PTSA Monohydrate

The initial PTSA catalyst contained total of 122.2 g PTSA (i.e., 112.7 g PTSA plus 9.5 g fresh PTSA catalyst). It was understood that the lower catalyst layer contains 85% PTSA, 6% salicylic acid, and 9% alkylated salicylic acid products. Therefore, the 110.7 g lower catalyst layer would contain 94.1 g PTSA. The upper layer would contain 28.1 g PTSA (0.1632 mole), which is the difference between the starting mass of PTSA (122.2 g) and the remaining PTSA in the lower layer after the reaction. Accordingly, 2.94 g water could be added to the upper layer to convert 0.1632 mole PTSA into PTSA monohydrate.

Additional aspects, advantages and features of the invention are set forth in this specification, and in part will become apparent to those skilled in the art on examination of the following, or may be learned by practice of the invention. The inventions disclosed in this application are not limited to any particular set of or combination of aspects, advantages and features. It is contemplated that various combinations of the stated aspects, advantages and features make up the inventions disclosed in this application.

What is claimed is:

1. A process for preparing an alkylsalicylic acid, comprising:
reacting salicylic acid with an olefin having at least four carbon atoms at a temperature ranging from about 50° C. to about 200° C. in the presence of an arylsulfonic acid-containing catalyst, to produce an alkylsalicylic acid with a residual olefin of less than about 2 wt %, wherein the molar ratio of the olefin to salicylic acid is about 1:1 to about 1.3:1.

2. The process of claim 1, wherein the catalyst is para-toluene sulfonic acid, xylene sulfonic acid, naphthalene sulfonic acid, phenol sulfonic acid, or combinations thereof.

3. The process of claim 2, wherein the catalyst is para-toluene sulfonic acid, and wherein the catalyst is at least 95% pure, contains 0-5% water, and contains 0-5% sulfuric acid.

4. The process of claim 1, wherein the olefin is added to the reaction in two or more portions over a course of about 30 minutes to about 20 hours under the reaction temperature.

5. The process of claim 4, wherein the olefin is added to the reaction in two or more portions over the course of about 2 to about 6 hours, under reaction temperature.

6. The process of claim 1, wherein the olefin is an α-olefin containing 4 to 24 carbon atoms, or a mixture thereof.

7. The process of claim 1, wherein the molar ratio of the arylsulfonic acid to salicylic acid is about 0.5:1 to about 1.5:1.

8. The process of claim 1, wherein the temperature ranges from about 90° C. to about 110° C.

9. The process of claim 1, further comprising, after the reacting step:
adding an organic solvent to the reaction mixture to separate a catalyst layer from the reaction mixture layer, wherein at least 70% of the arylsulfonic acid catalyst is recovered through the separation.

10. The process of claim 9, wherein the organic solvent is light naphtha, heptane, or octane.

11. The process of claim 9, further comprising:
drying the catalyst contained in the separated catalyst layer; and
adding the dried catalyst back into the reaction.

12. The process of claim 9, further comprising:
precipitating the remaining catalyst contained in the reaction mixture layer by adding water in an amount sufficient to form an arylsulfonic acid monohydrate precipitant; and
recovering the arylsulfonic acid monohydrate precipitant by filtration.

13. The process of claim 12, further comprising:
drying the precipitated catalyst; and
adding the dried catalyst back into the reaction.

14. The alkylsalicylic acid prepared by the process of claim 1.

15. A process for preparing an alkylsalicylic acid, comprising:
reacting salicylic acid with an olefin having at least four carbon atoms at a temperature ranging from about 50° C. to about 200° C. in the presence of para-toluene sulfonic acid, to produce an alkylsalicylic acid with a total acid number no less than 90.

16. The process of claim 15, wherein the para-toluene sulfonic acid contains less than 5% water.

17. The process of claim 15, wherein the para-toluene sulfonic acid contains no more than 0.5% water.

18. The process of claim 15, wherein the catalyst is prepared by a process comprising:
reacting toluene with a sulfonating agent at a temperature ranging from about 0° C. to about 200° C.; and
removing water from the reaction mixture.

19. The process of claim 15, wherein the sulfonating agent is concentrated sulfuric acid with the reaction temperature ranging from about 50° C. to about 200° C., sulfur trioxide with the reaction temperature ranging from about 0° C. to about 30° C., or fuming sulfuric acid with the reaction temperature ranging from about 0° C. to about 70° C.

20. The process of claim 15, wherein the olefin is added to the reaction in two or more portions over a course of about 2 to about 6 hours under the reaction temperature.

21. The process of claim 15, wherein the molar ratio of the para-toluene sulfonic acid to salicylic acid is about 0.5:1 to about 1.5:1.

22. The process of claim 15, wherein the temperature ranges from about 90° C. to about 110° C.

23. The alkylsalicylic acid prepared by the process of claim 15.

24. The alkylsalicylic acid of claim 23, wherein the acid number is no less than 135.

25. The alkylsalicylic acid of claim 23, wherein the residual olefin is no more than about 2.0 wt %.

26. The process of claim 4, wherein the olefin is added continuously.

27. The process of claim 20, wherein the olefin is added continuously.
